(19) United States
(12) Patent Application Publication
(10) Pub. No.: US 2016/0342763 A1
(43) Pub. Date: Nov. 24, 2016

(54) HEALTH MONITORING ASSIST SYSTEM

(71) Applicant: OMRON CORPORATION, Kyoto-shi, Kyoto (JP)

(72) Inventors: Hirotaka WADA, Nara (JP); Keiichi OBAYASHI, Nara (JP); Ayako KOKUBO, Kyoto (JP)

(73) Assignee: OMRON Corporation, Kyoto-shi, Kyoto (JP)

(21) Appl. No.: 15/111,020

(22) PCT Filed: Dec. 26, 2014
(86) PCT No.: PCT/JP2014/084650

§ 371 Date: Jul. 12, 2016

(30) Foreign Application Priority Data
Jan. 14, 2014 (JP) 2014-004141

(51) Int. Cl. G06F 19/00; A61B 5/022; A61B 5/00; A61B 5/11

(52) U.S. Cl. CPC G06F 19/3431; A61B 5/1118; A61B 5/022; A61B 5/7275; A61B 5/742; G06F 19/3481

(57) ABSTRACT

A health monitoring assist system includes: a unit that acquires time-series data of an activity index that indicates an amount of an activity conducted by a subject; a unit that acquires time-series data of a biological index measured from, and relating to health of, the subject; an estimation unit that estimates an amount of time lag between a change in the activity index and a resultant change appearing in the biological index based on a result of comparison between the time-series data of the activity index and the time-series data of the biological index; and a unit that provides the subject with assist information that indicates that an effect caused by the activity of the subject is apparent in a value of the biological index after the amount of time lag from a time point when the activity was conducted, based on an estimation result of the estimation unit.

### TECHNICAL FIELD

The present invention relates to a technique for assisting health monitoring of a user.

### BACKGROUND ART

Exercise such as walking is known to help reduce blood pressure, body weight, body fat, and abdominal girth, and to be effective in prevention or treatment of lifestyle-related diseases. However, engaging in a strenuous activity only for a brief period is pointless, and it is instead important to take moderate exercise continuously. Systems with a function of providing a graphical representation of changes in blood pressure, body weight, and the like, or a function of giving advice on the exercise or lifestyle habits have hitherto been proposed, as means of keeping motivation up to continue with the exercise. For example, in the system of PTL 1, time-series data of body weight, BMI, body fat percentage, number of steps, calories burned, highest blood pressure, lowest blood pressure, pulse, blood glucose level, urinary glucose level, cholesterol level, etc. are displayed in one chart, so that the user him/herself can check the data by comparing changes in several biological indexes.

### CITATION LIST

### Patent Literature

PTL 1: Japanese Patent Application Laid-open No. 2004-230099

### SUMMARY OF INVENTION

### Technical Problem

From the results of analysis of data of a large number of test subjects, the applicants of the present invention have found out that: (1) The effect of exercise (such as reduction in blood pressure or body weight) does not appear immediately, and there is a certain time lag between the period in which exercise is taken and the period in which its effect appears; (2) The amount of time lag can be relatively long, ranging from several days to several weeks; and (3) The amount of time lag differs from person to person. However, conventional systems do not take account of such delay characteristics of the effect of exercise (in particular, the difference in time lag among individuals) at all. Even if provided with a chart of time-series data of the number of steps and body weight as in the system of PTL 1, for example, if the user is not familiar with the delay characteristics of the effect of exercise, the user may wrongly think that the exercise has had no effect, or that there is no causal relationship between the number of steps and the body weight, and may lose the motivation for the exercise. Also, systems that give advice on exercise or lifestyle habits may present wrong advice unless proper consideration is taken in regard of the difference in time lag among individuals.

Note that, while the discussion above casts light on the relationship between exercise and its effect, a similar time lag may be observed between the period when activities other than exercise (such as eating and sleeping) that may affect health (biological indexes) are conducted and the period when its effects appear.

The present invention has been made in view of the circumstances described above, its object being to provide a technique for enabling health monitoring assistance that takes account of a time lag between an activity and appearance of its effect. A further object of the present invention is to provide a technique for presenting a causal relationship between an activity conducted by a subject and its effect in an accessible manner despite possible differences among individuals in the amount of time lag between activity and effect, so as to keep the person motivated to carry on the activity.

### Solution to Problem

The invention according to claim 1 is

a health monitoring assist system, including:

an activity index data acquisition unit that acquires time-series data of an activity index indicative of an amount of an activity conducted by a subject;

a biological index data acquisition unit that acquires time-series data of a biological index measured from, and relating to health of, the subject;

a time lag estimation unit that estimates an amount of time lag between a change in the activity index and a resultant change appearing in the biological index based on a result of comparison between the time-series data of the activity index and the time-series data of the biological index; and

an information presentation unit that provides the subject with assist information that indicates that an effect caused by the activity of the subject is apparent in a value of the biological index after the amount of time lag from a time point when the activity was conducted, based on an estimation result of the time lag estimation unit.

A “biological index” here refers to an indicator relating to a person's health measured from a person or samples collected from a person. Examples of biological indexes include blood pressure, pulse, body weight, body fat percentage, body fat mass, muscle percentage, muscle mass, abdominal girth, BMI, cholesterol level, blood glucose level, urinary glucose level, body temperature, and so on. An “activity” is a movement or behavior that may affect health (biological index). While exercises such as walking, jogging, and swimming are regarded as typical activities, other physical activities in everyday life other than exercises (such as commuting and household chores), as well as sleeping, resting, eating, taking/administering medicine, and taking supplements, are also considered as activities. An “activity index” indicates a quantified amount of an activity, and may be, for example, a number of steps, distance, duration of exercise, amount of exercise (calories burned), amount of activity (intensity of exercise multiplied by time), sleeping duration, duration or number of rest, calories taken, dosage of medicine, amount of supplements taken, and so on.

According to the invention set forth in claim 1, an amount of time lag between a change in an activity index and a resultant change appearing in a biological index is estimated, and based on this estimation result, a subject is provided with assist information that indicates that an effect caused by the activity of the subject is apparent in a value of the biological index after the amount of time lag from a time point when the activity was conducted. Therefore, health monitoring assistance that takes into account a time lag between activity and effect is made possible. In addition, since the time lag between activity and effect is estimated with the use of activity index and biological index data of the subject him/herself, a causal relationship between an activity conducted by the subject and its effect can be presented in an accessible manner despite the difference in the amount of time lag among individuals, so that the person can be kept motivated to carry on the activity.

The invention according to claim 2 is

a health monitoring assist system according to claim 1, wherein the assist information includes information indicative of the amount of time lag.

According to the invention set forth in claim 2, the subject can be made aware of his intrinsic physiological characteristics that show how much the appearance of an effect of his activity is delayed. Accordingly, the subject can realize what changes in the biological index he/she should compare with the increase or decrease of the activity index, and check the fruits of past activities or notice deterioration of the biological index caused by laziness. Also, the subject will be able to monitor his health (such as planning an exercise, setting a weight loss goal, or deciding dosage of medicine or timing of taking medicine) in consideration of his own physiological characteristics.

The invention according to claim 3 is

a health monitoring assist system according to claim 1 or 2, wherein the assist information includes a graph plotting the time-series data of the activity index and shifted by the amount of time lag, and a graph plotting the time-series data of the biological index.

According to the invention set forth in claim 3, the subject can intuitively understand the causal relationship between an increase/decrease of the activity index and a change in the biological index by visually comparing two graphs.

The invention according to claim 4 is

a health monitoring assist system according to any one of claims 1 to 3, wherein the assist information includes information indicating a period, in a graph plotting the time-series data of the biological index, in which a positive change caused by an increase in the activity index is apparent in the biological index.

According to the invention set forth in claim 4, the subject can easily grasp that an increase in the activity index (e.g., past effort) directly leads to a positive change in the biological index (e.g., health improvement), so that the person can be motivated to carry out and continue to do the activity.

The invention according to claim 5 is

a health monitoring assist system according to any one of claims 1 to 4, wherein the assist information includes information indicating a period, in a graph plotting the time-series data of the biological index, in which a negative change caused by a decrease in the activity index is apparent in the biological index.

According to the invention set forth in claim 5, the subject can easily grasp that a decrease in the activity index (e.g., past laziness) directly leads to a negative change in the biological index (e.g., health deterioration). This way, the subject can be encouraged to reflect upon him/herself and reminded of the necessity of doing the activity continuously.

The invention according to claim 6 is

a health monitoring assist system according to any one of claims 1 to 5, wherein the time lag estimation unit evaluates intensity of a causal correlation between the time-series data of the activity index and the time-series data of the biological index with respect to a plurality of amounts of time lag, and selects an amount of time lag with which the intensity of the correlation is highest as the amount of time lag between the change in the activity index and the resultant change appearing in the biological index.

According to the invention set forth in claim 6, the amount of time lag between an increase/decrease in the activity index and a change in the biological index can be determined suitably.

The invention according to claim 7 is

a health monitoring assist system according to claim 6, wherein the time lag estimation unit evaluates the intensity of the causal correlation between the time-series data of the activity index and the time-series data of the biological index with the use of transfer entropy.

According to the invention set forth in claim 7, the intensity of the causal correlation between the activity index (cause) and the biological index (result) is evaluated with the use of transfer entropy, so that the amount of time lag between an increase/decrease in the activity index and a change in the biological index can be determined more suitably.

The invention according to claim 8 is

a health monitoring assist system according to any one of claims 1 to 7, wherein the information presentation unit presents the assist information to a terminal owned by the subject by wired or wireless connection.

According to the invention set forth in claim 8, the subject can check the causal relationship between an increase/decrease in the activity index and a change in the biological index in the terminal the person owns, which is convenient. Health monitoring assist services using the so-called cloud computing may also be provided by adopting a configuration in which the terminal and the health monitoring assist system are connected via a wide-area network such as the Internet.

The invention according to claim 9 is

a terminal capable of communicating with the health monitoring assist system according to claim 8 by wired or wireless connection, including:

a request unit that transmits a request for assist information to the health monitoring assist system together with information for identifying the subject who owns the terminal; and

a display unit that displays the assist information received from the health monitoring assist system.

According to the invention set forth in claim 9, the subject can check the causal relationship between an increase/decrease in the activity index and a change in the biological index in the terminal the person owns, which is convenient. Health monitoring assist services using the so-called cloud computing may also be enjoyed by adopting a configuration in which the terminal and the health monitoring assist system are connected via a wide-area network such as the Internet. Since there is no need for the terminal to store activity index data and biological index data, or to calculate the time lag or produce assist information, the terminal can be configured simply, and the terminal cost can be reduced.

The invention according to claim 10 is

a terminal according to claim 9, further including a data transmission unit that transmits data of an activity index of an activity conducted by the subject and/or data of a biological index measured from the subject to the health monitoring assist system.

According to the invention set forth in claim 10, data collection and management in the health monitoring assist system can be achieved easily.

The invention according to claim 11 is

a terminal according to claim 9 or 10, further including a measurement unit that measures the activity index.

According to the invention set forth in claim 11, a single terminal can function as an activity index measurement device as well as a device that provides the health monitoring assist information, which is convenient.

The invention according to claim 12 is

a terminal according to claim 9 or 10, further including a measurement unit that measures the biological index.

According to the invention set forth in claim 11, a single terminal can function as a biological index measurement device as well as a device that provides the health monitoring assist information, which is convenient.

The invention according to claim 13 is

a program that makes a computer function as:

an activity index data acquisition unit that acquires time-series data of an activity index indicative of an amount of an activity conducted by a subject;

a biological index data acquisition unit that acquires time-series data of a biological index measured from, and relating to health of, the subject;

a time lag estimation unit that estimates an amount of time lag between a change in the activity index and a resultant change appearing in the biological index based on a result of comparison between the time-series data of the activity index and the time-series data of the biological index; and

an information presentation unit that provides the subject with assist information that indicates that an effect caused by the activity of the subject is apparent in a value of the biological index after the amount of time lag from a time point when the activity was conducted, based on an estimation result of the time lag estimation unit.

According to the invention set forth in claim 13, a system that estimates an amount of time lag between a change in an activity index and a resultant change appearing in a biological index, and, based on this estimation result, provides a subject with assist information that indicates that an effect caused by the activity of the subject is apparent in a value of the biological index after the amount of time lag from a time point when the activity was conducted, can be realized. This system can provide health monitoring assistance that takes into account a time lag between activity and effect. In addition, since the amount of time lag between activity and effect is estimated with the use of activity index and biological index data of the subject him/herself, a causal relationship between an activity conducted by the subject and its effect can be presented in an accessible manner despite the difference in the amount of time lag among individuals, so that the person can be kept motivated to carry on the activity.

The invention according to claim 14 is

a program that makes a terminal capable of communicating with the health monitoring assist system according to claim 8 by wired or wireless connection function as:

a request unit that transmits a request for assist information to the health monitoring assist system together with information for identifying the subject who owns the terminal; and

a display unit that displays the assist information received from the health monitoring assist system.

According to the invention set forth in claim 14, the subject can check the causal relationship between an increase/decrease in the activity index and a change in the biological index in the terminal the person owns, which is convenient. Health monitoring assist services using the so-called cloud computing may also be enjoyed by adopting a configuration in which the terminal and the health monitoring assist system are connected via a wide-area network such as the Internet. Since there is no need for the terminal to store activity index data and biological index data, or to calculate the amount of time lag or produce assist information, the terminal can be configured simply, and the terminal cost can be reduced.

### Advantageous Effects of Invention

The present invention can provide health monitoring assistance that takes into account a time lag between activity and effect. Also, with the present invention, a causal relationship between an activity conducted by a subject and its effect can be presented in an accessible manner despite the difference among individuals in the amount of time lag between activity and effect, so that the person can be kept motivated to carry on the activity.

### BRIEF DESCRIPTION OF DRAWINGS

FIG. 1 A function block diagram of a health monitoring assist system.

FIG. 2 Configuration examples of the health monitoring assist system.

FIG. 3 Configuration examples of the health monitoring assist system.

FIG. 4 An example of accumulated data of the number of steps and blood pressure in a data storage unit.

FIG. 5 A flowchart showing the process flow of the health monitoring assist system.

FIG. 6 A diagram for explaining a process of estimating an amount of time lag.

FIG. 7 Charts for explaining a process of generating a user assisting graph.

FIG. 8 A flowchart showing the flow of a process of detecting a period of an apparent effect of exercise.

FIG. 9 A chart showing an example of a detected period of an apparent positive effect of exercise SP-posi.

FIG. 10 A chart showing an example of joining consecutive periods.

FIG. 11 A diagram showing one example of a user assisting graph.

FIG. 12 One example of a template stored in an assist message pattern memory unit.

FIG. 13 A display example of assist information provided to a user.

FIG. 14 One example of a result of data analysis of a test subject.

FIG. 15 One example of a result of data analysis of a test subject.

FIG. 16 One example of a result of data analysis of a test subject.

FIG. 17 A result of plotting test subjects data of about 2000 cases.

### DESCRIPTION OF EMBODIMENTS

As one preferable embodiment of the present invention, a health monitoring assist system that takes account of a time lag between an activity and its effect will be described, and more particularly, a customized health monitoring assist system that takes account of the difference in the time lag among individuals and can provide assist information in accordance with personal characteristics will be described. Below, the relationship between “exercise (walking)” and “hypotensive effect” will be described first as one example of an “activity” and its “effect”, followed by illustration of specific configurations for the presentation of causal relationships or amounts of time lag between activity and effect based on time-series data of the number of steps and blood pressure, and for provision of assist information suited to personal needs.

<Relationship Between Exercise and Hypotensive Effect>

Continuous aerobic exercise such as walking has long been known to be effective in preventing or treating hypertension. However, the relationship between the time point of exercise and the time period when the hypotensive effect owing to the exercise appears has never been determined quantitatively.

The applicants of the present invention conducted an analysis of data of a large number of test subjects based on a concept of information theory called “transfer entropy (TE)”, and confirmed from the result that in many cases there is a significant causal relationship between an increase/decrease in the amount of exercise (number of steps) and a change in the systolic blood pressure. In addition, the applicants came upon new findings that there is a certain time lag (delay) between an increase/decrease in the number of steps and a change in the blood pressure, and that the amount of time lag (amplitude of time lag) differs from person to person and can be as short as about 1 week and as long as about 8 weeks (2 months).

Transfer entropy is a measure or method of evaluating a causal relationship between two events X and Y in consideration of a time lag between the two events, according to which the amount of information transferred from event X to event Y after time s (entropy) is regarded as the degree of influence of event X (cause) on event Y (result) after time s (i.e., causal intensity). The correlation coefficient is a similar concept, but different from the transfer entropy in that the correlation coefficient only evaluates the intensity of relevance between event X and event Y (distribution similarity) and does not take account of the causal direction (which event is the cause and which is the result) and the time lag.

Given time-series data of events X and Y are expressed as x(t) and y(t), and probability density functions as P(x(t)) and P(y(t)), transfer entropy TE_{XY}(s) of the amount of time lag s, with event X being the cause and event Y being the result, can be calculated by the following formula:

$${TE}_{XY} ( s ) = [ {log}_{2} \frac{P ( y ( t + s ) , y ( t ) , x ( t ) ) \cdot P ( y ( t ) )}{P ( y ( t ) , x ( t ) ) \cdot P ( y ( t + s ) , y ( t ) )} ] \qquad [ Mathematical Formula 1 ]$$

where P(a, b) represents variables of joint probability density functions P(a) and P(b), and [*] represents time average of *.

As can be seen from the formula above, the transfer entropy can be calculated, if time-series data of two events X and Y and the amount of time lag s are provided. When there is a causal relationship between events X and Y such that X is the cause and Y is the result, TE_{XY}(s)>TE_{YX}(s) is established between TE_{XY}(s) and TE_{YX}(s) which is a value obtained through calculation with the cause and the effect being inverted. Therefore, by determining which of TE_{XY}(s) and TE_{YX}(s) is larger, we can determine whether or not there is a causal relationship and the direction of causality. The amount of time lag s between event X (cause) and event Y (result) can also be determined by calculating the transfer entropy TE_{XY}(s) with the value of s being varied, and by finding the value of s with which TE_{XY}(s) becomes the largest.

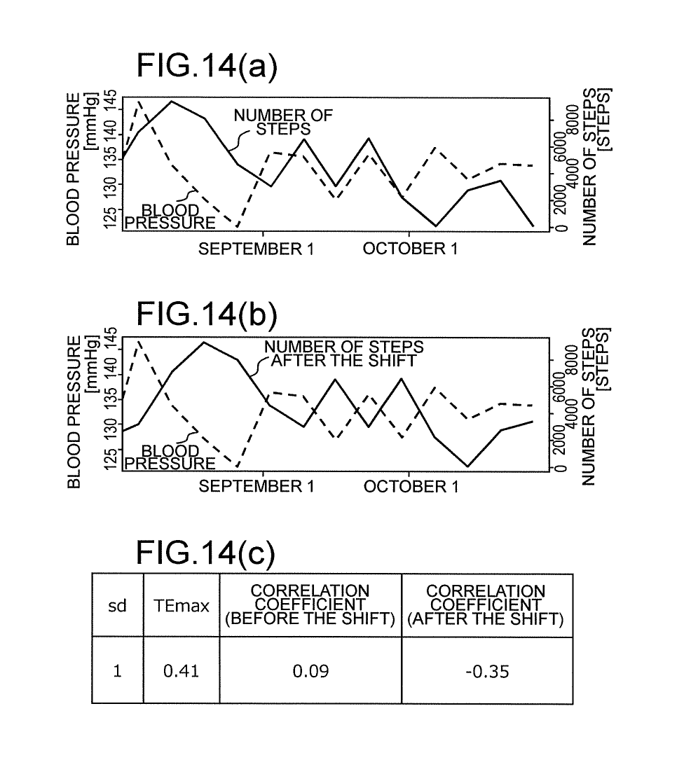

FIGS. 14(a) to 14(c) show a result of data analysis of a test subject. FIG. 14(a) is a graph obtained by plotting time-series data of the number of steps and systolic blood pressure, the horizontal axis representing time (week), and the vertical axis representing the number of steps (steps) and blood pressure value (mmHg). FIG. 14 (b) is a graph obtained by plotting time-series data of the number of steps with a shift by an amount sd (week), where sd is the amount of time lag when the transfer entropy is maximum, with the number of steps being the “cause” and the blood pressure value being the “effect”. Sd is one week in this example. From the graph of FIG. 14(b), one can see that an increase/decrease in the number of steps is reflected as a rise/drop of the blood pressure about a week later. FIG. 14 (c) shows an amount of time lag sd (week) when the transfer entropy is maximum, the maximum transfer entropy TEmax, the correlation coefficient between the number of steps and the blood pressure (i.e., the correlation coefficient between the number of steps and the blood pressure of FIG. 14(a)), and the correlation coefficient between the number of steps and the blood pressure after the shift by sd (i.e., the correlation coefficient between the number of steps and the blood pressure of FIG. 14 (b)). From this, one can see that there is clearly a negative correlation between the number of steps and the blood pressure after a week, i.e., the effect of exercise appears about a week later.

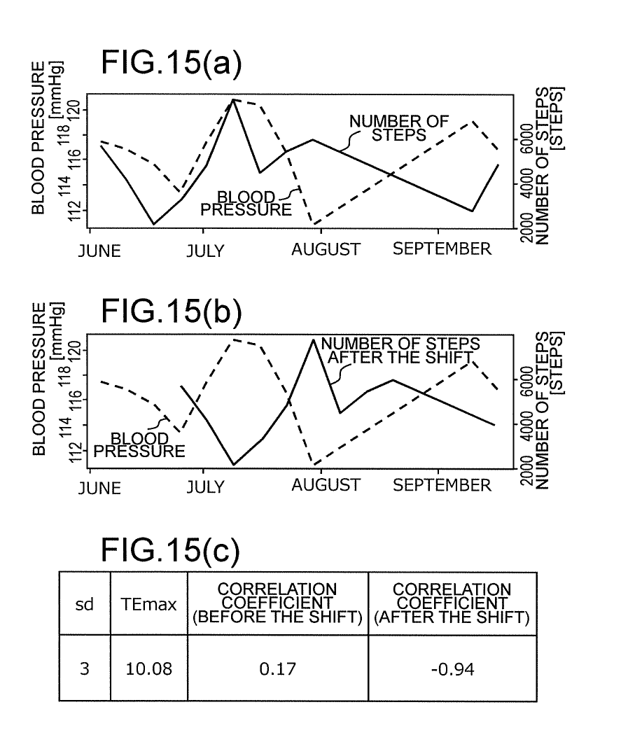

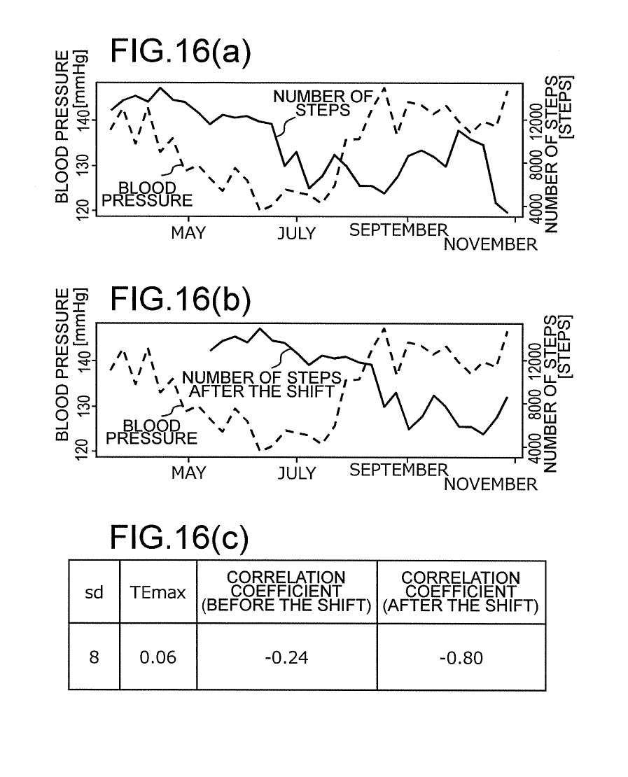

FIGS. 15(a) to 15(c) and FIGS. 16(a) to 16(c) show results of data analysis of different test subjects. In the case of FIGS. 15(a) to 15(c), the effect of the exercise appears with a delay of about 3 weeks, while the effect of the exercise appears with a delay of about 8 weeks in the case of FIGS. 16(a) to 16(c).

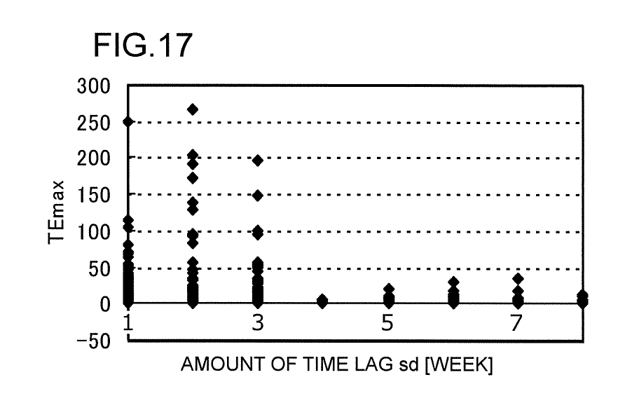

FIG. 17 is a result of plotting test subjects data of about 2000 cases. The horizontal axis represents the amount of time lag sd [week] while the vertical axis represents the maximum transfer entropy TEmax. It can be seen that the amount of time lag sd differs from person to person and can be as short as about 1 week and as long as about 8 weeks (2 months).

### <System Configuration>

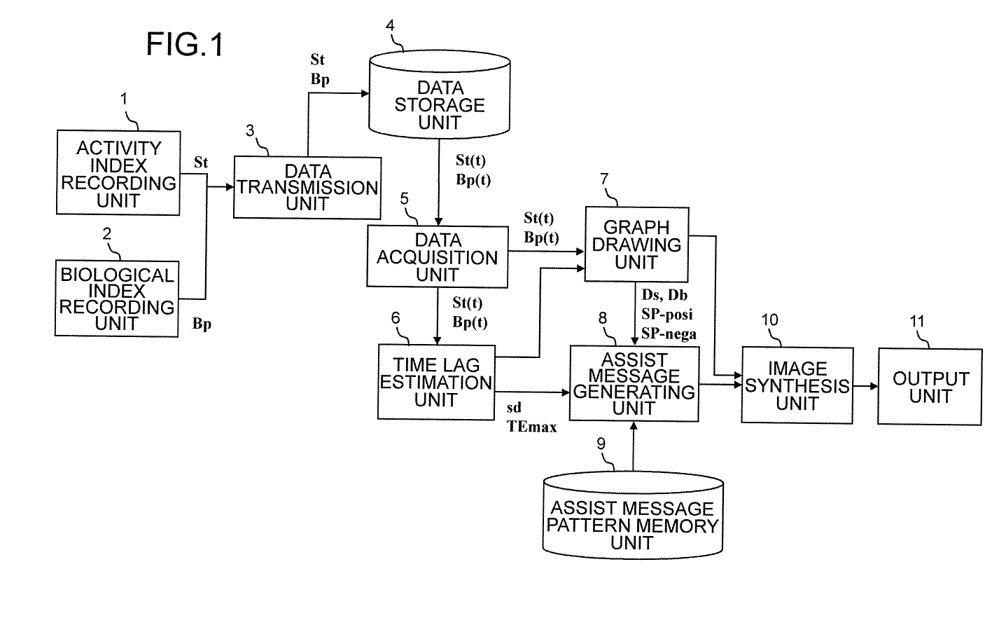

FIG. 1 is a function block diagram illustrating the overall configuration of the health monitoring assist system according to the embodiment (hereinafter also referred to simply as “system”).

This system includes function blocks such as an activity index recording unit 1, a biological index recording unit 2, a data transmission unit 3, a data storage unit 4, a data acquisition unit 5, a time lag estimation unit 6, a graph drawing unit 7, an assist message generating unit 8, an assist message pattern memory unit 9, an image synthesis unit 10, an output unit 11, and so on. These function blocks can be configured by one device, or a plurality of devices that can communicate with each other by a wired or wireless connection (specific examples of device configurations will be described later).

The activity index recording unit 1 is a function block that records an activity index (the number of steps in this embodiment) that indicates the amount of an activity conducted by a subject. The unit may be configured such that activity index values are manually input. Preferably, however, the activity index recording unit 1 may be configured with a measurement device such as a passometer or activity tracker so that the activity index values can be automatically measured and recorded.

The biological index recording unit 2 is a function block that records a biological index (blood pressure in this embodiment) measured from the subject. This unit may also be configured such that biological index values are manually input, but preferably, the biological index recording unit 2 may be configured with a measurement device such as a sphygmomanometer so that the biological index values can be automatically measured and recorded.

The data transmission unit 3 is a function block that transmits the activity index data recorded by the activity index recording unit 1 and biological index data recorded by the biological index recording unit 2 to the data storage unit 4 to be registered there. In this embodiment, data St of the number of steps of one day and data Bp of systolic blood pressure of each day are registered in the data storage unit 4.

The data storage unit 4 is a database for storing and managing, in a time-series manner, the activity index data and biological index data received via the data transmission unit 3. In this embodiment, time-series data St(t) of the number of steps and time-series data Bp(t) of the systolic blood pressure are stored. If this system is to be used by a plurality of users, the data need to be collected and stored for each of different users. In this case, the time-series data St(t) and Bp(t) may be managed in association with user IDs for distinguishing one user from another.

The data acquisition unit 5 is a function block that acquires the time-series data St(t) and Bp(t) from the data storage unit 4 to be used for data analysis and generation of assist information. In FIG. 1, one function block 5 represents an activity index data acquisition unit that acquires the time-series data of the activity index and a biological index data acquisition unit that acquires the time-series data of the biological index.

The time lag estimation unit 6 is a function block that estimates an amount of time lag between a change in the activity index and a resultant change appearing in the biological index based on a result of comparison between the time-series data of the activity index and the time-series data of the biological index acquired by the data acquisition unit 5. The time lag estimation unit 6 of this embodiment evaluates transfer entropy between the number of steps (cause) and the blood pressure (effect), using the time-series data St(t) of the number of steps and the time-series data Bp(t) of the blood pressure, and determines an amount of time lag sd, with which a maximum value of transfer entropy TEmax is achieved.

The graph drawing unit 7, assist message generating unit 8, assist message pattern memory unit 9, image synthesis unit 10, and output unit 11 are a group of function blocks that constitute an information presentation unit that provides assist information regarding health monitoring to a subject based on the estimation result of the time lag estimation unit 6. The assist information is a piece of information that shows the subject, in an accessible manner, that the effect caused by an activity of the person (walking) is apparent in a biological index (blood pressure) value with a time lag sd from a time point when the activity was conducted. In this embodiment, the assist information is provided in the form of a graph and an assist message. The graph drawing unit 7 generates a graph based on the time-series data St(t) and Bp(t) of the number of steps and blood pressure, and on the information such as the amount of time lag sd, maximum value of transfer entropy TEmax, and so on that are calculated at the time lag estimation unit 6. The assist message generating unit 8 generates an assist message based on the information such as the amount of time lag sd and the maximum value of transfer entropy TEmax that are calculated at the time lag estimation unit 6, an amount of change in the number of steps Ds, an amount of change in the blood pressure Db, and periods of an apparent effect of exercise SP-posi and SP-nega that are calculated at the graph drawing unit 7, with the use of a message template registered in the assist message pattern memory unit 9. The thus generated graph and assist message are integrated by the image synthesis unit 10 and output to a display device or external terminal by the output unit 11. Specific processes of generating assist information and specific examples of graphs and assist messages will be described later.

<Example of System Configuration>

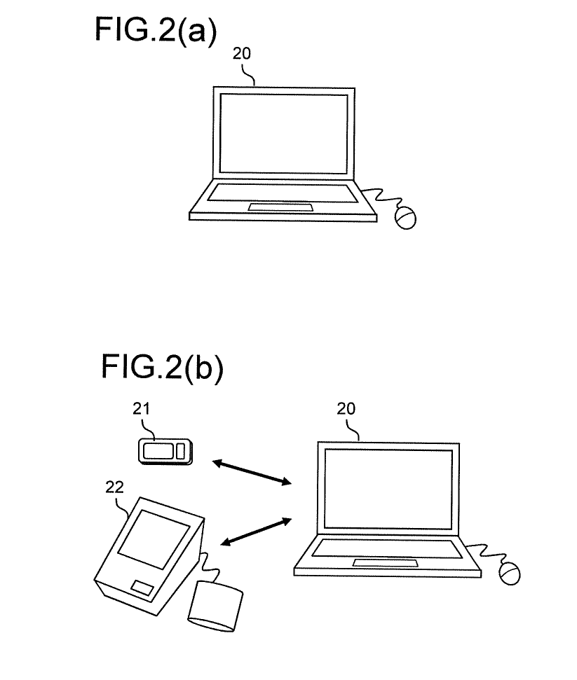

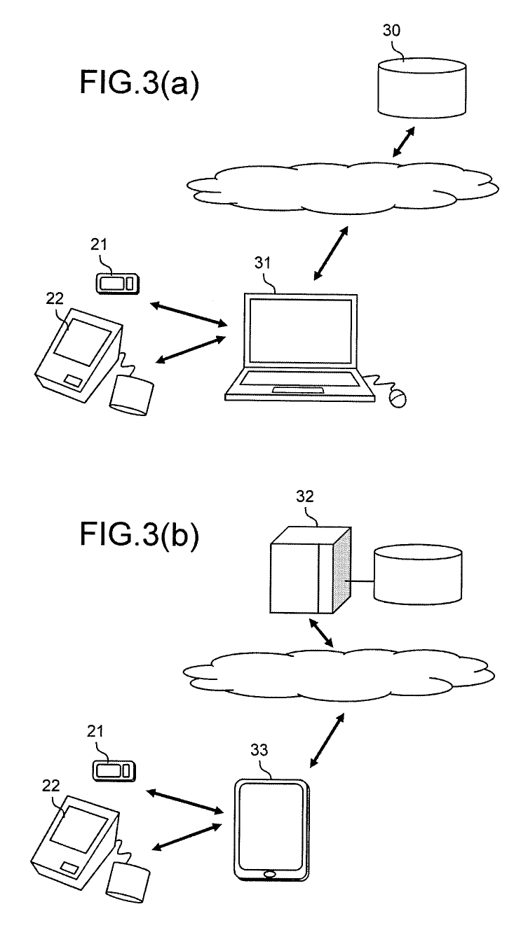

The health monitoring assist system of FIG. 1 can adopt various system configurations. Specific examples of system configurations are shown in FIG. 2(a) to FIG. 3(b).

FIG. 2(a) is one example of the health monitoring assist system configured with one device 20. This device 20 can be configured with a general-purpose personal computer that has a CPU (central processing unit), a main memory device (memory), an auxiliary memory device (hard disc, semiconductor disc, and the like), an input device (keyboard, mouse, touchscreen, and the like), a display device (liquid crystal monitor and the like), and a communication IF. The device 20 may also be configured as other equipment that offers the same or similar functions as those of personal computers such as a tablet terminal, smart phone, or PDA (personal digital assistant). Alternatively, the device 20 may be configured with a dedicated machine incorporating a single board computer therein. In the configuration of FIG. 2(a), a user operates an input device to input activity indexes and biological indexes, and assist information is output to a display device.

FIG. 2(b) is an example in which a passometer 21 and a sphygmomanometer 22 are used in combination with the device 20. Data measured with the passometer 21 and sphygmomanometer 22 are transmitted to the device 20 by a wired connection (e.g., via USB) or wirelessly (e.g., via Bluetooth® or WiFi). In this configuration, the activity index recording unit 1 and biological index recording unit 2 of FIG. 1 are configured by the passometer 21 and sphygmomanometer 22, respectively, and the functions of the data transmission unit 3 are realized by the data transmission functions incorporated in the passometer 21 and sphygmomanometer 22. The assist information may be output to the display device of the device 20, or transmitted to the passometer 21 or sphygmomanometer 22 and output to the display unit of the passometer 21 or sphygmomanometer 22.

FIG. 3(a) is one example of cloud computing, in which the data storage unit 4 of FIG. 1 is configured by an online storage 30. In the configuration of FIG. 3(a), a terminal 31 operated by a subject has the functions of the data transmission unit 3, data acquisition unit 5, time lag estimation unit 6, and information presentation unit of FIG. 1, and executes the uploading of the data measured with the passometer 21 and sphygmomanometer 22, downloading of the time-series data, and generation and display of the assist information. The terminal 31 may be configured by a personal computer, tablet terminal, smart phone, or other dedicated machine, similarly to the device 20 shown in FIGS. 2(a) and 2(b). If the passometer 21 or sphygmomanometer 22 is accessible to the Internet, the data may be uploaded to the online storage 30 directly from the passometer 21 or sphygmomanometer 22.

FIG. 3(b) also shows an example of cloud computing. The difference from FIG. 3(a) is that a cloud server 32 serves the functions of the data storage unit 4, data acquisition unit 5, time lag estimation unit 6, and information presentation unit of FIG. 1. A terminal 33 operated by a subject need only include functions such as a request unit that requests assist information from the cloud server 32, and a display unit that displays the assist information received from the cloud server 32. Identification of the subject may be achieved, for example, by including a user ID in the request for assist information, or by user ID authentication. The configuration of FIG. 3(b) is preferable when health monitoring assist service is provided by an application of a tablet terminal or smart phone, in particular, since the terminal 33 requires less resources (such as data capacity and operation performance) and can be configured simply.

<System Operation>

Next, specific examples of processes performed by this system, of analyzing data and of generating and displaying assist information, will be described.

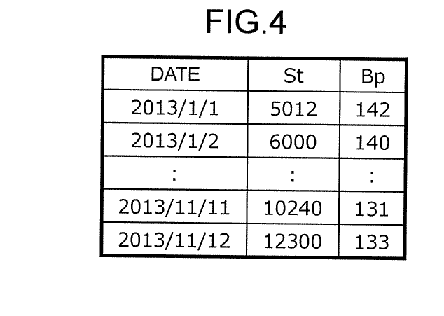

As a precondition, the data storage unit 4 already contains data of the number of steps and blood pressure a subject has recorded over a certain period of time (of, for example, one month or more). FIG. 4 shows one example of a data structure of the data storage unit 4. The information on the number of steps St and blood pressure Bp is stored together with the information on the measurement dates.

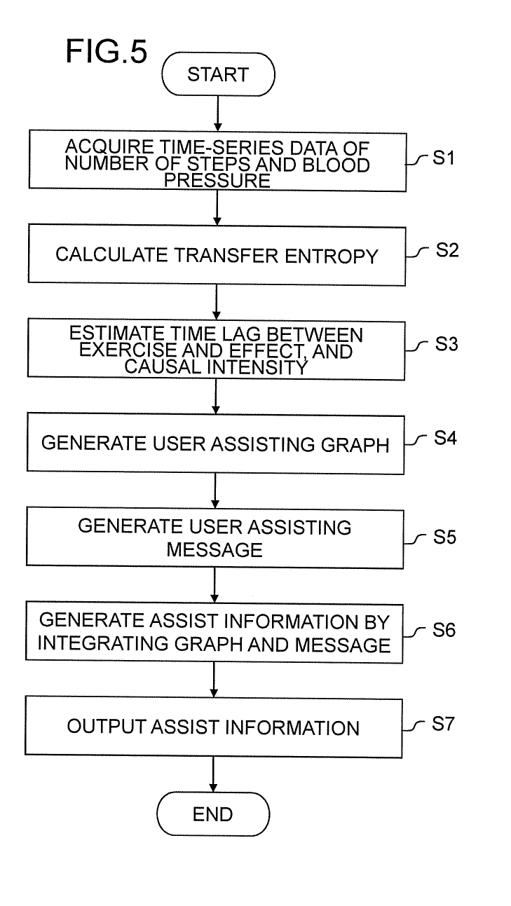

The process flow of this system will be explained along the flowchart of FIG. 5.

At step S1, the data acquisition unit 5 acquires time-series data St(t) of the number of steps and time-series data Bp(t) of the systolic blood pressure from the data storage unit 4. Here, the data of all the periods stored in the data storage unit 4 may be acquired, or, the data of a predetermined period (for example, of the immediate months n) may be acquired.

At step S2, the time lag estimation unit 6 calculates a transfer entropy value TEsb(s), with the number of steps St being the cause and the blood pressure Bp being the effect, as well as a transfer entropy value TEbs(s), with the blood pressure Bp being the cause and the number of steps St being the effect, with respect to a plurality of amounts of time lag s=0, 1, . . . , sm. Here, the unit of amount of time lag s is “day”, as is the time unit with which the number of steps St and blood pressure Bp are recorded. “sm” is a preset maximum number of days after which the effect of exercise could possibly appear. Here, sm=60 (days).

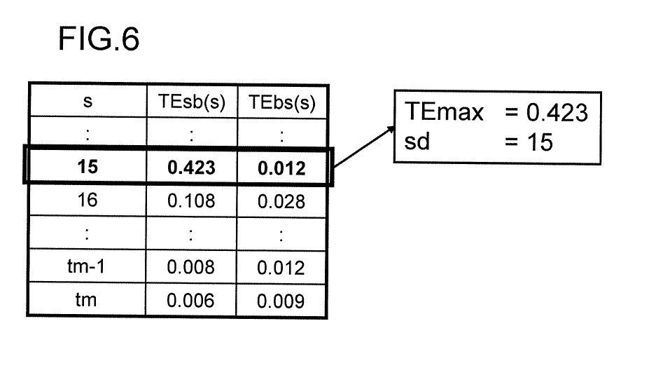

At step S3, the time lag estimation unit 6 defines the largest one of the transfer entropy values TEsb(s) calculated at step S2 that satisfy TEsb(s)>TEbs(s) (namely, of those with the direction of cause and effect being from the “number of steps” to the “blood pressure”) as TEmax, and records the amount of time lag sd with this maximum value. This maximum transfer entropy value TEmax is defined as causality intensity between the number of steps and the blood pressure. For example, if the calculation at step S2 has produced the results shown in FIG. 6, the maximum transfer entropy value TEmax and the amount of time lag sd are determined as 0.423 and 15, respectively. Through the process described above, the delay characteristics (amount of time lag sd being 15 days) of the effect of exercise intrinsic to this subject can be estimated.

Next, the process goes on to the step of generating assist information by the information presentation unit. In this embodiment, a user assisting graph is generated at step S4, and a user assisting message is generated at step S5. Each of these steps will be described in detail below.

(Step S4) Generation of a user assisting graph

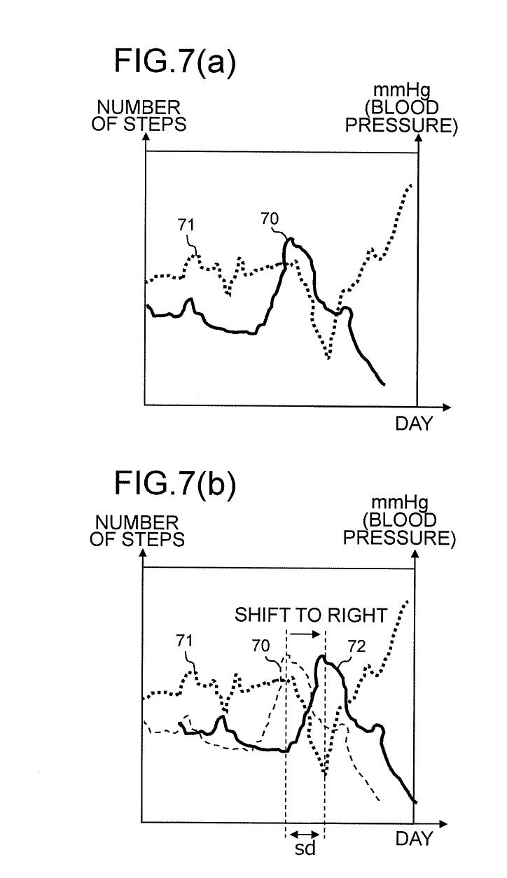

First, the graph drawing unit 7 receives time-series data St(t) of the number of steps and time-series data Bp(t) of the systolic blood pressure from the data acquisition unit 5, and creates a line graph, with the horizontal axis representing time (days) and the vertical axis representing the number of steps (steps) and blood pressure (mmHg). FIG. 7(a) is one example of the graph, the line 70 representing the number of steps and the line 71 representing the blood pressure.

Next, the graph drawing unit 7 shifts the line 70 to the right by the amount of time lag sd determined at step S3 (alternatively, the line 71 of the blood pressure may be shifted to the left by sd). FIG. 7(b) is one example of the graph after the shift, the line 72 representing the number of steps shifted to the right by sd. Hereinafter, such a graph in which one of the lines has been shifted by the amount of time lag sd as shown in FIG. 7(b) will be referred to as “time-adjusted graph of number of steps and blood pressure”.

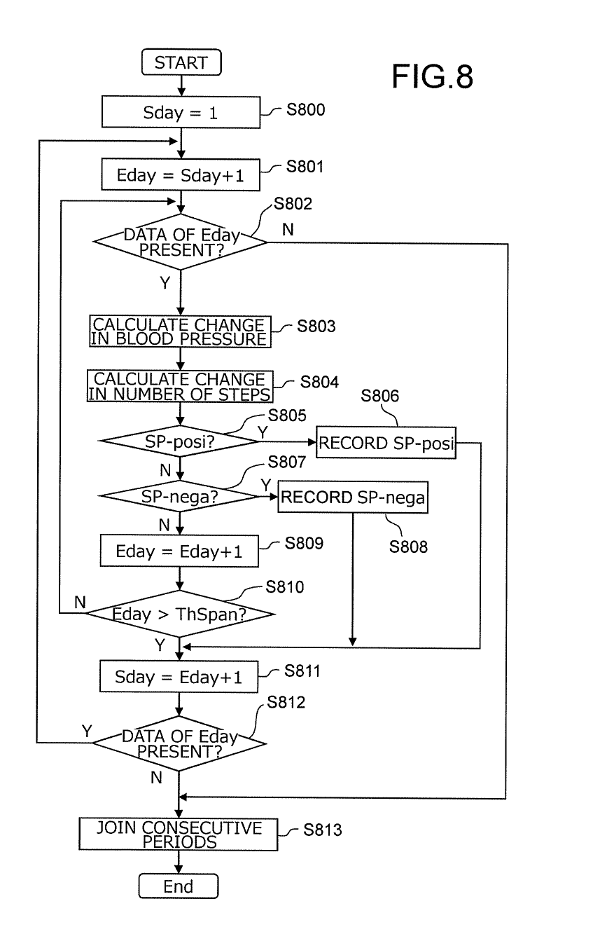

Next, the graph drawing unit 7 detects a period of an apparent positive effect of exercise SP-posi and a period of an apparent negative effect of exercise SP-nega from the time-adjusted graph of number of steps and blood pressure. Here, the “period of an apparent positive effect of exercise” refers to a period in the time-adjusted graph of number of steps and blood pressure where the number of steps increases while the blood pressure decreases, and the “period of an apparent negative effect of exercise” refers to a period in the graph where the number of steps decreases while the blood pressure increases. There can be various approaches to detect the periods of apparent positive and negative effects of exercise, and FIG. 8 shows one example.

In the following description, Sday and Eday represent variables indicating the date of starting calculation and the date of ending calculation, respectively, and Db and Ds represent variables indicating the change in blood pressure and the change in number of steps, respectively. Bp(t) and St(t) respectively represent a blood pressure value and a number of steps on a particular day t (the number of steps St(t) having been shifted by the amount of time lag sd). Also, it is assumed that suitable values are preset for the following thresholds.

ThSpan: Maximum period (of, e.g., 7 days) for calculating the amount of change

ThDifBp: Differential value (positive value of, e.g., 3 mmHg) for determining that the blood pressure has risen/dropped

ThDifSt: Differential value (positive value of, e.g., 1000 steps) for determining that the number of steps has increased/decreased

Once the process of FIG. 8 starts, the graph drawing unit 7 assigns 1 to the calculation start day Sday (step S800), and assigns Sday+1 to the calculation end day Eday (step S801). When there are data of the number of steps and blood pressure for the calculation end day Eday, the process goes to step S803, while, if there are no data of the number of steps and blood pressure, the process goes to step S813 (step S802).

At steps S803 and S804, the graph drawing unit 7 calculates the amount of change in the blood pressure Db and the amount of change in the number of steps Ds between the calculation start day Sday and the calculation end day Eday by the following equations:

Db=Bp(Eday)−Bp(Sday)

Ds=St(Eday)−St(Sday)

Successively, a period of an apparent positive effect of exercise SP-posi is determined (step S805). More specifically, if the amount of change in the blood pressure Db satisfies a condition “Db<−1×ThDifBp”, and the amount of change in the number of steps Ds satisfies a condition “Ds>ThDifSt” (step S805: Yes), the graph drawing unit 7 records the period from Sday to Eday as SP-posi (step S806), and the process goes to step S811. If Db and Ds fail to satisfy the conditions above (step S805: No), it is determined that no positive effect of exercise is apparent, and the process goes to step S807.

At step S807, a period of an apparent negative effect of exercise SP-nega is determined. More specifically, if the amount of change in the blood pressure Db satisfies a condition “Db>ThDifBp”, and the amount of change in the number of steps Ds satisfies a condition “Ds<−1×ThDifSt” (step S807: Yes), the graph drawing unit 7 records the period from Sday to Eday as SP-nega (step S808), and the process goes to step S811. If Db and Ds fail to satisfy the conditions above (step S807: No), it is determined that no negative effect of exercise is apparent, and the process goes to step S809.

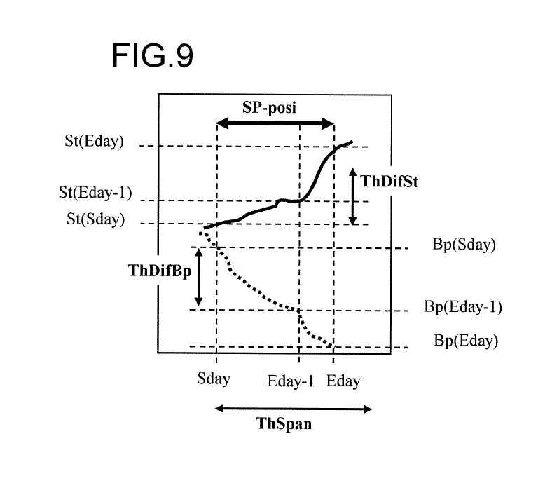

After that, Eday is incremented by 1 (step S809), and the steps from S802 to S809 are repeated (step S810) until Eday reaches ThSpan. That is, it is determined whether or not the period qualifies as the period of an apparent effect of exercise each time as the length of the period is extended by one day (the shortest being 1 day and the longest being ThSpan). Thereby, not just day-to-day changes in the number of steps and blood pressure but the trend of macro changes within a relatively long period can be evaluated, so that the possibility of missing out a period in which the effect of exercise is apparent is reduced. One example in which a period of an apparent effect of exercise is detected by changing the length of the period will be described with reference to FIG. 9.

FIG. 9 shows an example of detecting a period of an apparent positive effect of exercise SP-posi. In FIG. 9, the number of steps St(t) is increasing, while the blood pressure Bp(t) is decreasing. However, the period from Sday to Eday−1 is not determined as SP-posi, because the amount of change in the number of steps Ds=St(Eday−1)−St(Sday) does not satisfy the condition Ds>ThDifSt. However, in the process of checking the period from Sday to Eday afterwards, the amount of change in the blood pressure Db=Bp(Eday−1)−Bp(Sday) satisfies “Db<−1×ThDifBp” as well as the amount of change in the number of steps Ds=St(Eday)−St (Sday) satisfies the condition “Ds>ThDifSt”, so that the period from Sday to Eday is detected as a period of an apparent positive effect of exercise SP-posi.

When the determination process of a period with the calculation start day Sday as a starting point is complete, the graph drawing unit 7 renews the calculation start day Sday (step S811), by Sday=Eday+1. When there are data of the number of steps and blood pressure for the renewed Sday, the process goes back to step S801 and the process step is repeated, while, if there are no data of the number of steps and blood pressure, the process goes to step S813 (step S812).

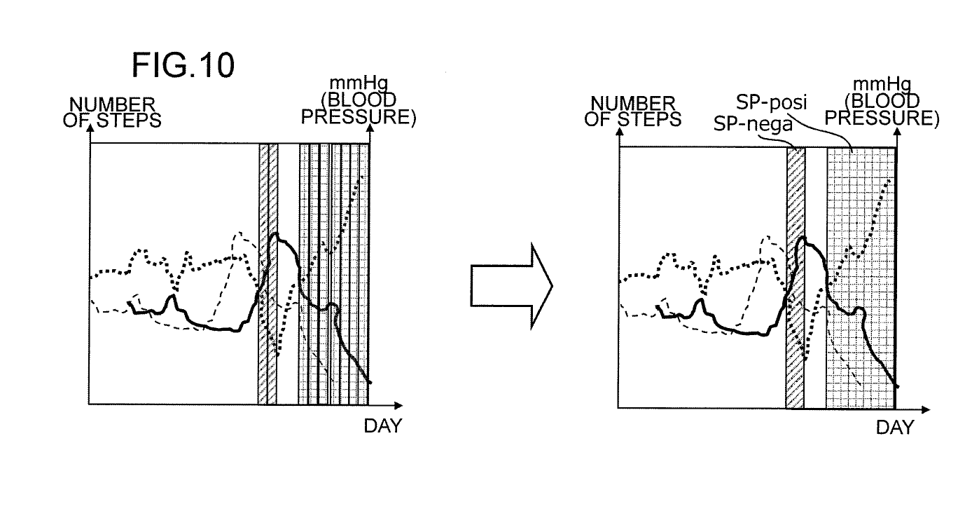

At step 3813, if any of the plurality of detected periods of apparent effect of exercise are consecutive, the graph drawing unit 7 joins these consecutive periods. A period of an apparent positive effect of exercise SP-posi and a period of an apparent negative effect of exercise SP-nega are not joined with each other. FIG. 10 shows an example of joining consecutive periods. The graph on the left side in FIG. 10 shows detected SP-posi and SP-nega, while the graph on the right side shows SP-posi and SP-nega after consecutive periods have been joined together. Adjacent periods only may be joined together, or, if the periods adjoin each other with a very short interval (of, e.g., one day to several days), they may be regarded as consecutive periods and joined together.

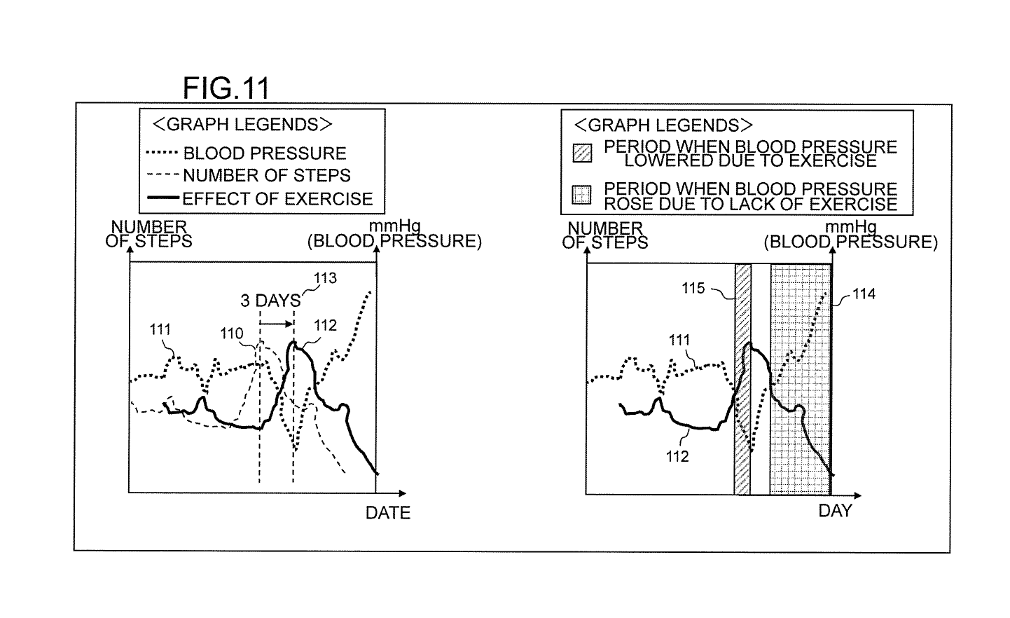

The graph drawing unit 7 generates a user assisting graph to be presented to the user, based on the information obtained through the processes described above. FIG. 11 is one example of a user assisting graph. In this example, two graphs are shown. The graph on the left side includes a line representing the number of steps 110, plotting the time-series data of the number of steps, and a line representing the blood pressure 111, plotting the time-series data of the blood pressure, as well as a line representing the effect of exercise 112 obtained by shifting the number-of-steps line 110 by an amount of time lag, and information 113 indicating the amount of time lag (3 days in this example). The graph on the right side shows a period of an apparent positive effect of exercise 114 and a period of an apparent negative effect of exercise 115 drawn over the blood pressure 111 and the effect-of-exercise line 112. There can be a plurality of periods of an apparent effect of exercise, all of which may be drawn over the graph, or only a typical one (such as the longest one) may be drawn over the graph. Captions for various elements may be set suitably as required.

(Step S5) Generation of a user assisting message

At step S5, the assist message generating unit 8 generates a user assisting message based on the results of the process from step S2 to step S4. Any information may be presented as a user assisting message, as long as it is useful for the user to monitor his health or continue to take exercise. In this embodiment, as one example, the user assisting message includes two types of information, the first message including an evaluation of the exercise carried out by the user and/or advice for future exercise, and the second message including delay characteristics (amount of time lag) of the effect of exercise of the user in question.

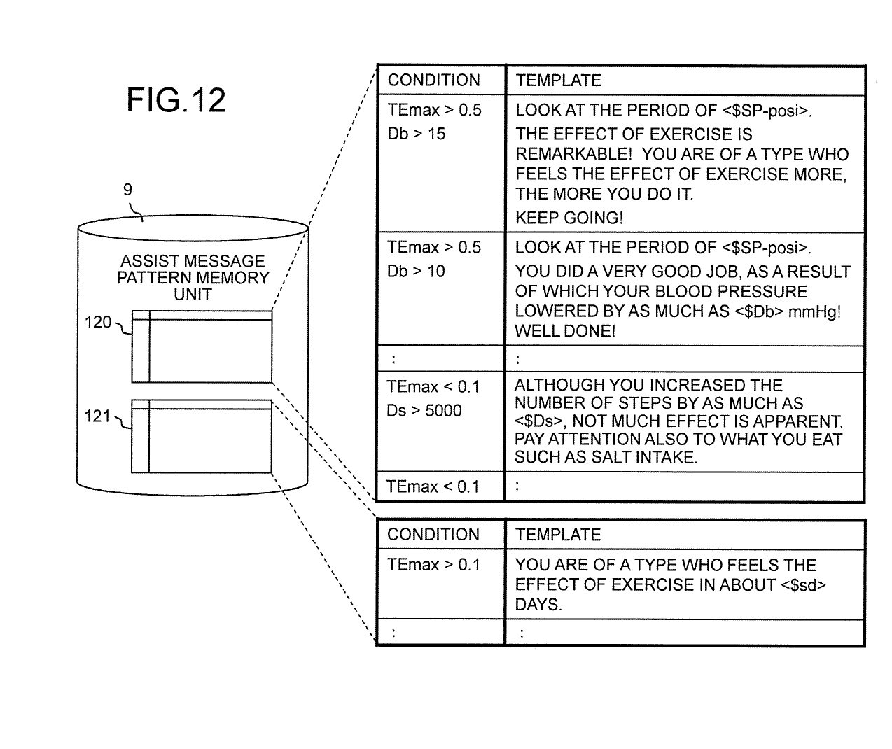

FIG. 12 shows one example of a template stored in an assist message pattern memory unit 9. The assist message pattern memory unit 9 includes a first table 120 in which a plurality of templates for the first message are registered, and a second table 121 in which a plurality of templates for the second message are registered. These templates may be registered in advance in this system, or may be added, edited, and deleted by a system administrator or a user. Each of the templates is registered corresponding to a condition with which the template can be selected. The condition for selection may be set, for example, using any of the values obtained through the process from step S2 to step S4, such as causal intensity (maximum transfer entropy) TEmax, number of steps St, blood pressure Bp, change in the number of steps Ds, change in the blood pressure Db, and amount of time lag sd. In the example of FIG. 12, the template for the first message is corresponded to a selection condition that is a combination of causal intensity TEmax, and change in the blood pressure Db or change in the number of steps Ds. The template for the second message is corresponded to causal intensity TEmax as a selection condition. The string of characters between “<$” and “>” embedded in the template is the part replaced with the value obtained through the process from step S2 to step S4 when a user assisting message is generated.

For example, if the following values are obtained as a result of the process from step S2 to step S4:

SP-posi: From Feb. 5, 2013 to Feb. 12, 2013

TEmax: 0.53

sd: 3

Db: 14

Ds: 2050,

the assist message generating unit 8 selects the second template from the first table 120, and generates a first message that says:

“Look at the period from Feb. 5, 2013 to Feb. 12, 2013. You did a very good job, as a result of which the blood pressure lowered by as much as 14 mmHg. Well done!” At the same time, the first template is selected from the second table 121, and a second message is generated that says:

“You are of a type who feels the effect of exercise in about three days”.

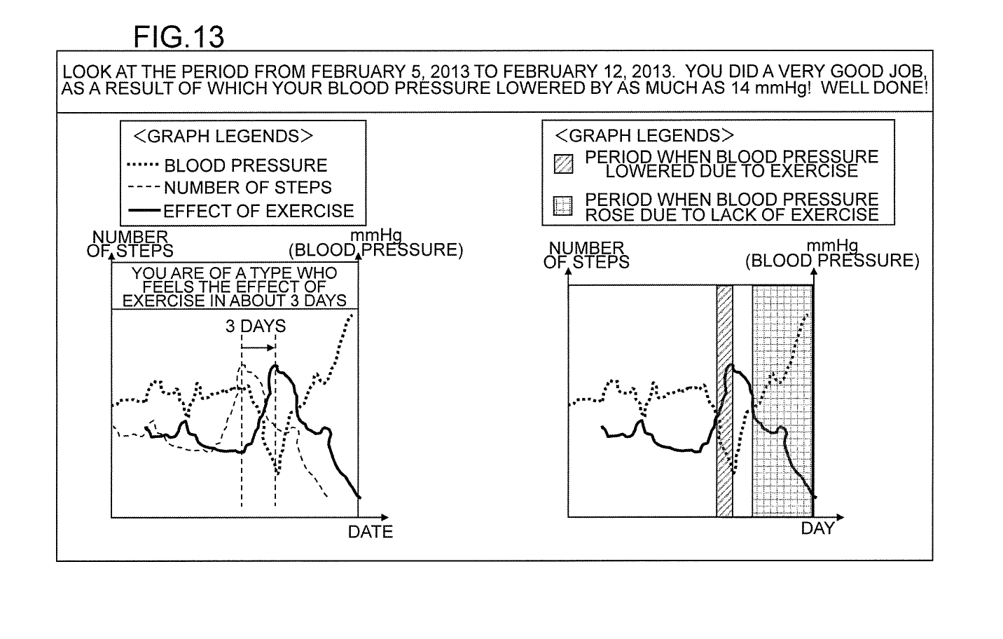

The thus generated user assisting message is integrated into a specified region of the user assisting graph by the image synthesis unit 10 (step S6) and output to a display device or external terminal by the output unit 11 (step S7). FIG. 13 is a display example of assist information. The first message is displayed in an upper part of the screen, while the second message is displayed in the upper part of the graph on the left side. The user, upon viewing such assist information, can realize the delay characteristics of the effect of exercise of his own, as well as intuitively understand and be convinced of the effect of the exercise.

<Advantages of this System>

The system described above can provide health monitoring assistance that takes into account a time lag between an activity and its effect. In particular, since the amount of time lag sd is estimated with the use of data of the number of steps and blood pressure of the user him/herself, a causal relationship between the exercise the user did and its effect can be presented in an accessible manner despite the difference in the amount of time lag sd among individuals, so that the user can be kept motivated to carry on the exercise.

The user can understand his intrinsic physiological characteristics, which is that the effect caused by the exercise appears with a delay of about 3 days, by looking at the screen shown in FIG. 13. Accordingly, the user can realize what changes in the blood pressure he/she should compare with the increase or decrease of the number of steps, and check the fruits of past exercises or notice deterioration of the blood pressure caused by laziness. Also, the user will be able to monitor his health (such as planning an exercise, setting a goal of the number of steps or blood pressure) in consideration of his own physiological characteristics.

The user can intuitively understand the causal relationship (negative correlation) between the increase/decrease in the number of steps and the change in the blood pressure by visually comparing the graph of the effect of exercise (line representing the number of steps after the shift) and the graph of the blood pressure in the screen shown in FIG. 13. Moreover, the display of a period of an apparent positive effect of exercise SP-posi allows the user to easily grasp that an increase in the number of steps (i.e., past effort) directly leads to a positive change in the blood pressure (i.e., a drop of blood pressure), so that the user can be motivated to carry out and continue to take the exercise. Moreover, the display of a period of an apparent negative effect of exercise SP-nega allows the user also to easily grasp that a decrease in the number of steps (i.e., past laziness) directly leads to a negative change in the blood pressure (i.e., a rise of blood pressure). This way, the user can be encouraged to reflect upon him/herself and reminded of the necessity of taking the exercise continuously.

### Variation Example

The configuration in the embodiment described above is shown only to illustrate one specific example of the present invention and not intended to limit the scope of the present invention. The present invention can adopt various specific configurations without departing from the technical concepts of the present invention.

For example, in the process of step S1 in FIG. 5, some data in the data storage unit 4 may be left out (missing). Data can be missing for various reasons, for example, some data may have been failed to be entered into the data storage unit 4, or the number of steps or blood pressure may have been failed to be measured, or some errors may have occurred in the measurement device or computer. Such missing data may be detrimental to the processing of time-series data. The data acquisition unit 5 therefore may carry out a process of complementing missing data using the preceding and following data. For example, a missing part of data may be created by linearly interpolating data points before and after the missing part, or by nearest neighbor interpolation (wherein missing data points are made the same as the preceding or following data point).

While time-series data by day was used in the embodiment described above, average values of the number of steps and blood pressure per week may be calculated, so that the time-series data by day can be converted to time-series data by week, and the causal relationship between exercise and effect may be analyzed using the time-series data by week. By using average values per week, missing data points and outliers are eliminated from the data, so that the reliability of the analysis can be expected to improve. The unit may not necessarily be week, but may be N days (N>1), or month.

Although “exercise (walking)” and “hypotensive effect” are given as examples of “activity” and “effect”, and “number of steps” and “blood pressure” are given as examples of “activity index” and “biological index” in the embodiment described above, the present invention can be applied to a wider range than this. Various other applications are possible such as, for example, analyzing a “weight loss effect” by an “exercise (walking)” from time-series data of the “number of steps” and “body weight”, analyzing a “weight loss effect” by a “physical activity” from time-series data of the “amount of exercise (calories burned) or amount of activity (metabolic equivalent of task multiplied by time)” and “body fat percentage”, and analyzing an “effect of medicine” by “taking medicine” from time-series data of the “dose of medicine” and “blood glucose level”.

### REFERENCE SIGNS LIST

1: Activity indicator recording unit
2: Biological marker recording unit
3: Data transmission unit
4: Data storage unit
5: Data acquisition unit
6: Time lag estimation unit
7: Graph drawing unit
8: Assist message generating unit
9: Assist message pattern memory unit
10: Image synthesis unit
11: Output unit
20: Device
21: Passometer
22: Sphygmomanometer
30: Online storage
31: Terminal
32: Cloud server
33: Terminal

1. A health monitoring assist system, comprising:
an activity index data acquisition unit that acquires time-series data of an activity index indicative of an amount of an activity conducted by a subject;
a biological index data acquisition unit that acquires time-series data of a biological index measured from, and relating to health of, the subject;
a time lag estimation unit that estimates an amount of time lag between a change in the activity index and a resultant change appearing in the biological index based on a result of comparison between the time-series data of the activity index and the time-series data of the biological index; and
an information presentation unit that provides the subject with assist information that indicates that an effect caused by the activity of the subject is apparent in a value of the biological index after the amount of time lag from a time point when the activity was conducted, based on an estimation result of the time lag estimation unit.

2. The health monitoring assist system according to claim 1, wherein the assist information includes information indicative of the amount of time lag.

3. The health monitoring assist system according to claim 1, wherein the assist information includes a graph plotting the time-series data of the activity index and shifted by the amount of time lag, and a graph plotting the time-series data of the biological index.

4. The health monitoring assist system according to claim 1, wherein the assist information includes information indicating a period, in a graph plotting the time-series data of the biological index, in which a positive change caused by an increase in the activity index is apparent in the biological index.

5. The health monitoring assist system according to claim 1, wherein the assist information includes information indicating a period, in a graph plotting the time-series data of the biological index, in which a negative change caused by a decrease in the activity index is apparent in the biological index.

6. The health monitoring assist system according to claim 1, wherein the time lag estimation unit evaluates intensity of a causal correlation between the time-series data of the activity index and the time-series data of the biological index with respect to a plurality of amounts of time lag, and selects an amount of time lag with which the intensity of a correlation is highest as the amount of time lag between the change in the activity index and the resultant change appearing in the biological index.

7. The health monitoring assist system according to claim 6, wherein the time lag estimation unit evaluates the intensity of the causal correlation between the time-series data of the activity index and the time-series data of the biological index with use of transfer entropy.

8. The health monitoring assist system according to claim 1, wherein the information presentation unit presents the assist information to a terminal owned by the subject by wired or wireless connection.

9. A terminal capable of communicating with the health monitoring assist system according to claim 8 by wired or wireless connection, comprising:
a request unit that transmits a request for assist information to the health monitoring assist system together with information for identifying the subject who owns the terminal; and
a display unit that displays the assist information received from the health monitoring assist system.

10. The terminal according to claim 9, further comprising a data transmission unit that transmits data of an activity index of an activity conducted by the subject and/or data of a biological index measured from the subject to the health monitoring assist system.

11. The terminal according to claim 9, further comprising a measurement unit that measures the activity index.

12. The terminal according to claim 9, further comprising a measurement unit that measures the biological index.

13. A non-transitory computer readable storage medium storing a program that makes a computer function as:
an activity index data acquisition unit that acquires time-series data of an activity index indicative of an amount of an activity conducted by a subject;
a biological index data acquisition unit that acquires time-series data of a biological index measured from, and relating to health of, the subject;
a time lag estimation unit that estimates an amount of time lag between a change in the activity index and a resultant change appearing in the biological index based on a result of comparison between the time-series data of the activity index and the time-series data of the biological index; and
an information presentation unit that provides the subject with assist information that indicates that an effect caused by the activity of the subject is apparent in a value of the biological index after the amount of time lag from a time point when the activity was conducted, based on an estimation result of the time lag estimation unit.

14. A non-transitory computer readable storage medium storing a program that makes a terminal capable of communicating with the health monitoring assist system according to claim 8 by wired or wireless connection function as:
a request unit that transmits a request for assist information to the health monitoring assist system together with information for identifying the subject who owns the terminal; and
a display unit that displays the assist information received from the health monitoring assist system.
